(19) United States
(12) Patent Application Publication
(10) Pub. No.: US 2018/0179596 A1
(43) Pub. Date: Jun. 28, 2018

(54) HISTONE MODIFICATION AGENTS FOR CANCER TREATMENT

(71) Applicant: MOR RESEARCH APPLICATIONS LTD., Tel Aviv (IL)

(72) Inventors: Smadar AVIGAD, Tel Aviv (IL); Isaac YANIV, Tel Aviv (IL); Lital SELA-TZURIANO, Tel Aviv (IL)

(21) Appl. No.: 15/739,961

(22) PCT Filed: Jun. 24, 2016
(86) PCT No.: PCT/IL2016/050676

§ 371 Date: Dec. 26, 2017

Related U.S. Application Data
(60) Provisional application No. 62/183,768, filed on Jun. 24, 2015.

(51) Int. Cl. C12Q 1/6886; A61K 38/50; A61K 45/06; C07K 14/47; A61K 31/603; G01N 33/574

(52) U.S. Cl. CPC C12Q 1/6886; A61K 38/50; A61K 45/06; C07K 14/4702; A61K 31/603; G01N 2333/91057; C12Q 2600/106; C12N 2310/14; C12Q 2600/156; C12Q 2600/158; G01N 33/57484

(57) ABSTRACT

Described herein are methods of cancer diagnosis through monitoring the presence and activity of the KANSL1 gene, and particularly the effects of KANSL1 overexpression on specific histone acetylation. Cancer treatment with histone acetyltransferase inhibitors and deacetylase agents is also described.

### CROSS REFERENCE TO RELATED APPLICATIONS

Benefit is claimed to U.S. Provisional Patent Application No. 62/183,768, filed Jun. 24, 2015, the contents of which are incorporated by reference herein in their entirety.

### FIELD

This disclosure relates to methods of cancer diagnosis through monitoring the presence and activity of the KANSL1 gene, and particularly the effects of KANSL1 overexpression on specific histone acetylation. Cancer treatment with histone acetyltransferases inhibitors and deacetylase agents is also described.

### BACKGROUND

Histone acetylation is one of the important post-translational modifications regulated by histone acetyltransferases (HATs) and deacetylases (HDACs) (Shahbazian et al). Acetyltransferases are involved in many biological processes, such as transcriptional regulation, DNA repair, and cell cycle progression.

MOF (males absent on the first) is a member of the MYST family of HATs which catalyzes the acetylation of histone H4 Lys 16 (H4K16) (Taipale et al.). MOF is part of the NSL (nonspecific lethal) complex involved in global transcription regulation (Raja et al.). The NSL complex is an evolutionarily conserved multi-protein assembly consisting of at least MOF, KANSL1, KANSL2, KANSL3, WDRS, MCRS1, and PHF20 in mammals (Raja et al.). In mammals, loss of MOF leads to early embryonic lethality (Thomas et al.).

KANSL1 (KAT8 regulatory NSL complex subunit 1, also called MSL1V1 or KIAA1267) is a protein-coding gene. Its chromosomal location is 17q21.31. The human KANSL1 subunit consists of 1105 amino acid residues. It is predicted to be mostly unstructured; however its C terminus contains the PEHE domain, which interacts with the histone acetyl transferase (HAT) domain of MOF (Kadlec et al.). MOF-KANSL1 complex was found to be specifically required for the acetylation of K120 on TP53, a tumor suppressor protein, and regulates apoptosis independent of transcription (Li et al.).

Haploinsufficiency of KANSL1 is sufficient to cause the 17q21.31 microdeletion syndrome, a multisystem disorder characterized by intellectual disability, hypotonia and distinctive facial features (Zollino et al, Koolen et al.). Zollino et al. have identified loss-of function mutations in KANLS1 gene in 2 individuals with the 17q21.31 microdeletion syndrome that lack the deletion, indicating that 17q21.31 deletion syndrome is a monogenic disorder caused by haploinsufficiency of KANSL1.

Mutations in KANSL1 have been identified in Down syndrome patients diagnosed with acute megakaryoblastic leukemia (AMKL) (Yoshida et al.). However, to date, aberrant overexpression of KANSL1 has not been associated with a pathological condition.

Histone modifications and their modifiers hold great promise as therapeutic targets because, in contrast to genetic mutations, they are dynamic and potentially reversible. Therefore, a continuing need exists to identify histone modifying agents that can be targeted for particular disease treatments.

### SUMMARY

The current disclosure is directed to the discovery of a strong correlation between aberrant gain of copy number of the KANSL1 gene, aberrant KANSL1 overexpression, and cancer. Increased risk of cancer relapse correlated with KANSL1 overexpression is also described.

In view of these and other related discoveries, provided herein are methods for diagnosing a cancer, predicting a predisposition to a cancer and/or predicting an appropriate epigenetic therapy for a cancer, the methods include detecting KANSL1 expression and/or copy number in a sample from a subject, wherein a measurable increase in KANSL1 expression and/or copy number when compared with a control diagnoses, predicts the predisposition, and/or predicts the appropriate epigenetic therapy for the cancer in the subject.

Also described are methods of determining the prognosis and the therapy of a cancer by detecting KANSL1 copy number and/or expression in a sample from a subject diagnosed with the cancer, and wherein a significant increase in KANSL1 copy number and/or expression compared with a control indicates an increased probability that the subject will relapse, thereby suggesting the need for therapies known to inhibit cancer relapse.

Also provided are methods of treating a cancer by administering to a subject in need thereof a therapeutically effective amount of an inhibitor of KANSL1 activity, thereby treating the cancer.

The foregoing and other objects, features, and advantages will become more apparent from the following detailed description, which proceeds with reference to the accompanying figures.

### BRIEF DESCRIPTION OF THE DRAWINGS

FIG. 1 shows a graphic illustration of the smooth signal of chromosome 17q having gain of KANSL1 copy number (lanes 1 and 2) and a normal KANSL1 copy number (lane 3).

FIG. 2A shows RNA levels of KANSL1 measured by RQ-PCR in the ALL cohort (n=36) in KANSL1 copy number gain versus normal copy number. FIG. 2B shows RNA levels of KANSL1 measured by RQ-PCR in the NBL cohort (n=26) in KANSL1 copy number gain versus normal copy number.

FIG. 3 shows RNA levels of KANSL1 measured by RQ-PCR in the AML cohort (n=12) in KANSL1 copy number gain versus normal copy number.

FIGS. 4A and 4B show KANSL1 protein levels in ALL BM samples (n=19). FIG. 4A is a representative Western blot gel. FIG. 4B is a graphical illustration of the quantitated average protein expression.

FIG. 5 shows the difference in Histone H3 acetylation in ALL BM samples (n=27).

FIG. 6 shows the difference in Histone H4 acetylation in ALL BM samples (n=27).

FIGS. 7A and 7B show MOF protein levels in ALL BM samples (n=30). FIG. 7A is a representative Western blot gel. FIG. 7B is a graphical illustration.

FIGS. 8A and 8B show TP53 protein levels in ALL BM samples (n=22). FIG. 8A is a representative Western blot gel. FIG. 8B is a graphical illustration.

FIG. 9 shows total Histone H4 acetylation in NALM6 cells overexpressing KANSL1 (plasmid) verses a negative control (nc). Results obtained from 3 independent experiments.

FIG. 10 shows specific Histone H4 modifications in NALM6 cells overexpressing KANSL1 (plasmid) verses a negative control (nc).

FIG. 11 shows total Histone H3 acetylation in NALM6 cells overexpressing KANSL1 (plasmid) verses a negative control (nc). Results obtained from 3 independent experiments.

FIG. 12 shows specific Histone H3 modifications in NALM6 cells overexpressing KANSL1 (plasmid) verses a negative control (nc).

FIGS. 13A and 13B show MOF protein levels in NALM6 cell lines with (plasmid) and without (nc) KANSL1 plasmid. Results obtained from 3 independent experiments. FIG. 13A is a representative Western blot gel. FIG. 13B is a graphical illustration.

FIGS. 14A and 14B show TP53 protein levels in NALM6 cell lines with (plasmid) and without (nc) KANSL1 plasmid. Results obtained from 2 independent experiments. FIG. 14A is a representative Western blot gel. FIG. 14B is a graphical illustration.

FIG. 15 shows H3 acetylation levels following treatment with a HAT inhibitor, MG-149. Plasmid=overexpression of KANSL1; Plasmid+MG-149=overexpression of KANSL1+20 μM of MG-149 agent. NC=Negative Control; NC+MG-149=negative control (with regard to KANSL1 expression)+20 μM of MG-149 agent.

FIG. 16 shows RNA levels of KANSL1 measured by RQ-PCR in 27 primary ependymoma samples. A significant correlation between KANSL1 copy number gain and high expression levels was identified (p=0.0046).

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. In the attached sequence listing:

SEQ ID NOs 1 and 2 are forward and reverse primers (respectively) for detecting KANSL1 expression by RQ-PCR.

SEQ ID NOs 3 and 4 are forward and reverse primers (respectively) for detecting actin expression by RQ-PCR.

### DETAILED DESCRIPTION

### I. Abbreviations

ALL Acute lymphoblastic leukemia

AML Acute myeloid leukemia

BM Bone marrow

HAT Histone acetyltransferase

HDAC Histone deacetylase

NBL Neuroblastoma

### II. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art, and as appropriate for the particular condition and location under treatment. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Altered expression: Expression of a biological molecule (for example, mRNA or protein) in a subject or biological sample from a subject that deviates from expression of the same biological molecule in a subject or biological sample from a subject having normal or unaltered characteristics for the biological condition associated with the molecule. Normal expression can be found in a control, a standard for a population, etc. Altered expression of a biological molecule may be associated with a disease, for example the increased copy number and expression of KANSL1 is associated with certain forms of cancer. The term “associated with” includes an increased risk of developing the disease as well as the disease itself. Expression may be altered in such a manner as to be increased or decreased. The directed alteration in expression of mRNA or protein may be associated with therapeutic benefits. For example, decreased expression of KANSL1 to levels associated with normal genomic copy number.

Altered protein expression refers to expression of a protein that is in some manner different from expression of the protein in a normal (wild type) situation.

Controls or standards appropriate for comparison to a sample, for the determination of altered expression, include samples believed to express normally as well as laboratory values, even though possibly arbitrarily set, keeping in mind that such values may vary from laboratory to laboratory. Laboratory standards and values may be set based on a known or determined population value and may be supplied in the format of a graph or table that permits easy comparison of measured, experimentally determined values.

Antagonist: A molecule or compound that tends to nullify the action of another, or in some instances that blocks the ability of a given chemical to bind to its receptor or other interacting molecule, preventing a biological response. Antagonists are not limited to a specific type of compound, and may include in various embodiments peptides, antibodies and fragments thereof, and other organic or inorganic compounds (for example, peptidomimetics and small molecules). As understood herein, histone deacetylases are understood to be antagonists of histone acetyltransferases.

Antibody: A polypeptide ligand comprising at least a light chain or heavy chain immunoglobulin variable region, which specifically recognizes and binds an epitope of an antigen, such as the KANSL1 protein or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antisense inhibitor: Refers to an oligomeric compound that is at least partially complementary to the region of a target nucleic acid molecule to which it hybridizes. As used herein, an antisense inhibitor (also referred to as an “antisense compound”) that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulation expression. Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound.

Biological Sample: Any sample that may be obtained directly or indirectly from an organism, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organelles (such as mitochondria), organs, and/or extracts of tissues, cells (such as, fibroblasts, peripheral blood mononuclear cells, or muscle cells), organelles (such as mitochondria) or organs. A biological sample may also be a laboratory research sample such as a cell culture supernatant. The sample is collected or obtained using methods well known to those skilled in the art.

Cancer: The product of neoplasia is a neoplasm (a tumor or cancer), which is an abnormal growth of tissue that results from excessive cell division. A tumor that does not metastasize is referred to as “benign.” A tumor that invades the surrounding tissue and/or can metastasize is referred to as “malignant.” Neoplasia is one example of a proliferative disorder. A “cancer cell” is a cell that is neoplastic, for example a cell or cell line isolated from a tumor. Particular examples of cancer include ALL, AML, ependymoma, Ewing sarcoma, and neuroblastoma, and are examples of a “KANSL1-associated cancer”, as used herein.

Chemotherapeutic agent: An agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Control: A reference standard. A control can be a known value indicative of normal genomic copy number and expression of KANSL1. In particular examples a control sample is taken from a subject that is known not to have a disease or condition. In other examples, the control is taken from as subject who does have a disease or condition, such as ALL, but who does not have a gain in KANSL1 copy number. In other examples a control is taken from the subject being diagnosed, but at an earlier time point, either before disease onset or prior to or at an earlier time point in disease treatment.

A difference between a test sample and a control can be an increase or conversely a decrease. The difference can be a qualitative difference or a quantitative difference, for example a statistically significant difference, or a measurable increase, even if not statistically significant. In some examples, a measurable difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Detect: To determine if an agent (such as a signal or particular nucleotide nucleic acid probe, amino acid, or protein, for example a KANSL1 protein or nucleic acid) is present or absent. In some examples, this can further include quantification.

Determining expression of a gene product: Detection of a level of expression (for example protein or nucleic acid) in either a qualitative or a quantitative manner In one example, it is the detection of a KANSL1 gene product. Gene expression may be measured at the RNA level or the protein level and by any method known in the art, including Northern blot, RT-PCR (of all types, including qualitative and quantitative methods), Western blot, or in vitro, in situ, or in vivo protein activity assay(s).

Diagnosis: The process of identifying a disease or a predisposition to developing a disease, such as ALL, AML, ependymoma, Ewing sarcoma, or neuroblastoma, by its signs, symptoms, and results of various tests and methods, for example the methods disclosed herein. The conclusion reached through that process is also called “a diagnosis.” The term “predisposition” refers to an effect of a factor or factors that render a subject susceptible to a condition, disease, or disorder, such as cancer. In some examples, of the disclosed methods, testing is able to identify a subject predisposed to developing a condition, disease, or disorder. For example, a subject possessing a gain of KANSL1 copy number, and associated increased KANSL1 expression, will have a predisposition to developing certain types of cancers, as described herein.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity and type of the affliction, and the manner of administration of the compound.

Increased risk: As used herein “increased risk” of cancer refers to an increase in the statistical probability of developing cancer relative to the general population. In particular embodiments, a subject with a gain in KANSL1 copy number is said to have an increased risk of developing a cancer such as ALL.

Inhibiting protein activity: To decrease, limit, or block an action, function or expression of a protein. The phrase inhibit protein activity is not intended to be an absolute term. Instead, the phrase is intended to convey a wide-range of inhibitory effects that various agents may have on the normal (for example, uninhibited or control) protein activity. Inhibition of protein activity may, but need not, result in an increase in the level or activity of an indicator of the protein's activity. By way of example, this can happen when the protein of interest is acting as an inhibitor or suppressor of a downstream indicator. Thus, protein activity may be inhibited when the level or activity of any direct or indirect indicator of the protein's activity is changed (for example, increased or decreased) by at least 10%, at least 20%, at least 30%, at least 50%, at least 80%, at least 100% or at least 250% or more as compared to control measurements of the same indicator. Inhibition of protein activity may also be effected, for example, by inhibiting expression of the gene encoding the protein or by decreasing the half-life of the mRNA encoding the protein (e.g. through an RNAi agent).

Mammal: This term includes both human and non-human mammals Similarly, the term subject includes both human and veterinary subjects.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the compounds herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing or treating a disease: Preventing a disease refers to completely inhibiting the development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop. In particular examples however, treatment is similar to prevention, except that instead of complete inhibition the development, progression or relapse of the disease is inhibited or slowed.

RNA interference (RNA silencing; RNAi): A gene-silencing mechanism whereby specific double-stranded RNA (dsRNA) trigger the degradation of homologous mRNA (also called target RNA). Double-stranded RNA is processed into small interfering RNAs (siRNA), which serve as a guide for cleavage of the homologous mRNA in the RNA-induced silencing complex (RISC). The remnants of the target RNA may then also act as siRNA; thus resulting in a cascade effect. As used herein, an RNAi agent is any RNA agent that will promote the RNAi of a particular gene product, including particular miRNA sequences.

Sample: Encompasses a sample obtained from a subject, whether unfixed, frozen, or fixed in formalin or paraffin. In particular embodiments, a sample can be blood, serum, cerebrospinal fluid, bronchoalveolar lavage, pus, or a skin lesion.

Small molecule inhibitor: A molecule, typically with a molecular weight less than 1000, or in some embodiments, less than about 500 Daltons, wherein the molecule is capable of inhibiting, to some measurable extent, an activity of some target molecule. In particular examples, a small molecule inhibitor of KANSL1 associated HAT activity is MG-149.

Specific binding: Binding substantially only to a defined target. Thus a KANSL1 specific binding agent is an agent that binds substantially to KANSL1, and not to other molecules. Thus the term “specifically binds” refers, with respect to an antigen, to the preferential association of an antibody, in whole or part, with a cell or tissue bearing that antigen and not to cells or tissues lacking that antigen.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound. For example, a therapeutically effective amount of an active ingredient can be measured as the concentration (moles per liter or molar-M) of the active ingredient (such as a small molecule, peptide, protein, or antibody) in blood (in vivo) or a buffer (in vitro) that produces an effect.

### III. Overview of Several Embodiments

Provided herein are methods for diagnosing a cancer, predicting a predisposition to a cancer and/or predicting an appropriate epigenetic therapy for a cancer by detecting KANSL1 expression and/or copy number in a sample from a subject, such as a human or non-human subject, wherein a measurable increase in KANSL1 expression and/or copy number when compared with a control diagnoses the cancer, predicts the predisposition, and/or predicts the appropriate epigenetic therapy for the cancer in the subject.

In particular embodiments, the cancer is selected from the group consisting of ALL, AML, Ependymoma. Ewing sarcoma, and neuroblastoma.

In some embodiments, detecting KANSL1 expression comprises detecting KANSL1 RNA or protein in the sample.

In some embodiments, the methods of diagnosis, prognosis, and/or prediction indicate a need to inhibit NSL complex activity, and further include administration of an inhibitor of KANSL1 activity, such as an inhibitor of HAT activity of the NSL complex, an HDAC, or more specifically, includes MG-149.

Also described are methods of determining the prognosis and the therapy of a cancer in a subject by detecting KANSL1 copy number and/or expression in a sample from a subject diagnosed with the cancer, and wherein a significant increase in KANSL1 copy number and/or expression compared with a control indicates an increased probability that the subject will relapse.

In particular embodiments, the cancer is selected from the group consisting of ALL, AML, Ependymoma, Ewing sarcoma, and neuroblastoma.

In some embodiments, detecting KANSL1 expression includes detecting KANSL1 RNA or protein in the sample.

Further described herein are methods of treating a cancer, which include administering to a subject in need thereof a therapeutically effective amount of an inhibitor of KANSL1 activity, thereby treating the cancer.

In particular embodiments, the cancer being treated is selected from a group consisting of ALL, AML, Ependymoma. Ewing sarcoma, and neuroblastoma.

In some embodiments, the inhibitor blocks formation of the NSL transcriptional regulation complex. In other embodiments, the inhibitor blocks the HAT activity of the NSL complex.

In particular embodiments, the inhibitor specifically binds to KANSL1. In yet other embodiments, the inhibitor specifically binds to a non-KANSL1 member of the NSL complex, such as MOF.

In certain embodiments, the inhibitor transiently inhibits the expression of KANSL1, such as by an RNAi agent.

In other embodiments, the inhibitor is an HDAC.

In still further embodiments, the inhibitor of KANSL1 includes MG-149.

### IV. Detection of KANSL1 for Cancer Diagnosis and Prediction

Disclosed herein is the discovery that a gain in the genomic copy number of KANSL1 correlates with predisposition and development of cancer, including particular cancers such as ALL, AML, Ependymoma. Ewing sarcoma, and neuroblastoma. Gain in copy number results in increased KANSL1 mRNA and protein expression and can also result in increased expression of KANSL1-associated proteins MOF and TP53. Therefore, KANSL1 copy number gain, and by extension increased KANSL1 (as well as MOF and TP53) expression, can be used to diagnose certain cancers, predict a predisposition of certain cancers in a subject, and can be used to determine an appropriate epigenetic therapy.

In view of these discoveries, disclosed herein are methods of predicting the occurrence of cancer, such as ALL, AML, Ewing sarcoma, ependymoma, and neuroblastoma in a subject, and of determining an appropriate therapy for patients diagnosed with a cancer such as ALL, AML, neuroblastoma, Ewing sarcoma, ependymoma, or other KANSL-1 associated cancers. The methods include detecting KANSL1 copy number and/or KANSL1 expression, and comparing the level of expression detected to that in a control sample. A determination that the level of expression in the subject is measurably greater than that in the control indicates that the subject has or is predicted to have a tendency to develop a KANSL1-associated cancer, and will likely benefit from an epigenetic drug therapy that normalizes aberrant histone acetylation resultant from KANSL1 overexpression or which inhibits aberrant KANSL1-related histone acetylation. Such therapies include, but are not limited to. inhibition of KANSL1 expression, blockade of KANSL1 associated HAT complex formation, and/or administration of MG-149. In particular embodiments, such therapies include those treatments known to the art that are used to inhibit relapse in specific cancer types.

As described herein, a subject who has a gain in KANSL1 copy number is also more likely to experience cancer relapse. Therefore, also described herein are methods of determining cancer prognosis by predicting its relapse in the subject. This is done by detecting the KANSL1 copy number and/or expression in the subject and comparing the level detected with a control, wherein a measurable increase in KANSL1 copy number and/or expression in the subject indicates an increased probability that the subject will relapse.

The control to which a subject's KANSL1 copy number and/or expression is compared can vary, depending on several factors, including the subject and the method being practiced. In particular embodiments, the control is the level of KANSL1 copy number and/or expression in a sample obtained from at least one healthy subject that has not been diagnosed with a KANSL1-related cancer. In other embodiments, the control can be a sample from a subject who has a KANSL1-related cancer, but who has a normal KANSL1 copy number (e.g. such as in methods of predicting cancer relapse). In other embodiments, the control is a historical control or standard reference value or range of values (such as a previously tested control sample), for instance the average or otherwise collective level of a group of subjects who do not have a KANSL1-related cancer, or in particular examples are confirmed as having normal KANSL1 copy number. Control standards and values may be set based on a known or determined population value and may be supplied, for instance, in the form of a graph or table that permits easy comparison of measured, experimentally determined values.

KANSL1 copy number and/or KANSL1 (or MOF or TP53) expression can be determined by any method known to one of skill in the art, including standard DNA detection methodologies, including but not limited to RQ-PCR, SNP methodologies, oligonucleotide array, and FISH. In particular embodiments, KANSL1 (or MOF or TP53) expression is determined by measuring the level of expressed RNA in the sample. In other embodiments, expression is determined by measuring the level of expressed protein in the sample,

KANSL1 (or MOF or TP53) RNA expression can be measured by any method known in the art. In particular embodiments, RT-PCR or quantitative real time RT-PCR is used to measure the level of KANSL1 (or MOF or TP53) expression. In other embodiments, quantitative primer extension is used. In still other embodiments Northern blotting is used. The methods of transcriptional profiling contemplated herein include methods based on hybridization analysis of polynucleotides (such as using array, including micro-array, techniques) and methods based on sequencing of polynucleotides. In some examples, mRNA expression in a sample is quantified using Northern blotting or in situ hybridization (Parker & Barnes, Methods in Molecular Biology 106:247-283, 1999); RNAse protection assays (Hod, Biotechniques 13:852-4, 1992); and are inclusive of all PCR-based methods (such as RT-PCR). Alternatively, antibodies can be employed that can recognize specific nucleic acid duplexes.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes HPRT, GAPDH, β-actin, and 18S ribosomal RNA. A variation of RT-PCR is real time quantitative RT-PCR, which measures PCR product accumulation through a dual-labeled fluorogenic probe (e.g., TAQMAN® probe). Other real time RT PCR kits include the PerfeCTa® SYBR® green fastMix® Rox (Quanta Biosciences) RT PCR system. Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591.

In some embodiments, gene expression is identified or confirmed using a nucleic acid microarray-based technique.

Methods of detecting protein expression are also well known in the art, and KANSL1 (or MOF or TP53) protein expression can be measured by any such method, such as a standard immunoassay or variation thereof. Immunoassays are binding assays involving binding between antibodies and antigen. Many types and formats of immunoassays are known and all are suitable for detecting protein expression in the described methods. Examples of immunoassays include enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (1-RAP/FLAP).

In general, immunoassays involve contacting a sample suspected of containing a molecule of interest (such as KANSL1 protein) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specific ally bound antibody species, allowing only those antibodies specifically bound within the primary immune complexes to be detected.

The described immunoassays include quantifying the amount of a molecule of interest (such as KANSL1 protein). In general, the detection of immunocomplex formation is well known in the art and can be achieved through the application of numerous approaches. These methods are generally based upon the detection of a label or marker, such as any radioactive, fluorescent, biological or enzymatic tags or any other known label. In particular embodiments, wherein the proteins are detected by microarray format, and where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

### IV. Methods of Treatment using HAT Inhibitors and HDACs

KANSL1 is part of the NSL histone acetyltransferase (HAT) complex. The discovery herein of an association between gain of KANSL1 copy number, and associated increases in KANSL1, MOF, and TP53 expression, and cancer, including but not limited to ALL, AML, and neuroblastoma, indicates a role for aberrant increased KANSL1 activity in such cancers. This role is demonstrated herein by the observation in ALL patients of increased histone H3 acetylation, and increased acetylation at histone H4, lysine 16 (H4K16).

In view of these discoveries, compositions for treatment of KANSL1-associated cancers such as ALL, and methods of their use, are described herein. Such compositions include inhibitors of the KANSL1 HAT activity, which can be administered to a subject in need thereof, such as a patient diagnosed with ALL, AML, ependymoma, or neuroblastoma, or a patient identified as having gain in KANSL1 copy number, and who has not yet developed a KANSL1-associated cancer.

In particular embodiments, the compositions include inhibitors that block the formation of the NSL complex, such as antibodies, or fragments thereof that specifically recognize KANSL1 or a non-KANSL1 complex member such as MOF, and which prevent complex formation. Peptide fragments and small molecules serving similar functions are also contemplated. Other examples of HAT inhibitors for use in the described compositions and methods include neutralizing antibodies and small molecules that recognize the NSL complex or one or more members thereof and prevent HAT activity. For example, MOF a known member of the MYST family of HATs that is in part regulated by autoacetylation activity. Compositions that inhibit MOF autoacetylation are thus contemplated for use in the described methods.

In other embodiments, NSL HAT activity is inhibited by regulating the expression of one or more members of the NSL complex. Such regulation can be accomplished at any level of gene expression, whether transcription, RNA stability, or translation. In particular embodiments, KANSL1 expression is targeted by administering one or more RNAi agents to a subject, by any method known in the art of delivering such RNAi agents.

In still other embodiments, aberrant H3 and H416 acetylation in patients having gain of KANSL1 copy number can be treated by administering one or more histone deacetylase agents to a subject in need thereof. Such agents can be supplied exogenously (i.e. administered to a subject as a pharmaceutical/therapeutic agent as described herein) or produced intracellularly through standard expression systems.

The compositions and methods described herein can be used independently or as part of regimen for treating or inhibiting the development of KANSL1-associated cancers (such as ALL, AML, ependymoma, and neuroblastoma). Such treatment regimens combine use of the compositions described herein and additional biologic or chemotherapeutic agents known in the art for treating such cancers. In particular embodiments, the KANSL1 inhibitory agents are administered simultaneously with one or more additional treatment agents. In other embodiments, the KANSL1 inhibitory agents are administered in a sequence before or after a treatment course with the one or more additional agent.

The methods of treatment described herein can be performed independently of the described diagnostic methods or as part of an overall method of diagnosis and treatment. In such methods, the determination of a gain of KANSL1 copy number (e.g. through detection of a measurable increase in KANSL1 expression or direct measurement of KANSL1 copy number increase) indicates a need for the described methods of treatment in order to improve the prognosis or inhibit the development of the cancer. The steps of administering the HAT inhibitors or HDACs described herein follow the determination of a KANSL1 copy number gain.

Several inhibitors of histone acetyltransferase are known, and have been studied previously (Dekker et al). One such inhibitor is MG-149, a novel anacardic acid (6-pentadecylsalicylic acid) that demonstrates selectivity toward the MYST type of histone acetyltransferase (Tip60 and MOF) was used in embodiments, and is effectively suppresses hyperacetylation. Moreover, MG-149 inhibits the TP53 and the NF-kB pathways. It will be appreciated that in addition to MG-149 itself, the current disclosure contemplates use of functional derivatives of MG-149 that are substantially similar in structure to MG-149 so as to retain its histone acetyltransferase activity. Other inhibitors of histone acetyltransferase for use in the described methods include, but are not limited o Anacardic acid, Curcumin, Garcinol, CPTH2, and MB-3.

The following examples are provided to illustrate certain particular features and/or embodiments. These examples should not be construed to limit the disclosure to the particular features or embodiments described.

### EXAMPLES

### Example 1

### Increase of KANSL1 Copy Number in ALL, AML, and Neuroblastoma Patients

KANSL1 (KAT8 regulatory NSL complex subunit 1, also called MSL1V1 or KIAA1267) is a protein-coding gene located on 17q21.31. Though its translated protein is predicted to be mostly unstructured, its C terminus contains the PEHE domain, which interacts with the histone acetyl transferase (HAT) domain of MOF (Kadlec et al.). MOF-KANSL1 complex was found to be specifically required for the acetylation of K120 on TP53, a tumor suppressor protein, and regulates apoptosis independent of transcription (Li et al.). This example shows that KANSL1 genome copy number is increased in ALL, AML, and neuroblastoma patients.

To determine KANSL1 copy number, genome copy number analysis (Cytoscan HD SNP array, Affymetrix) was applied to DNA extracted from the following malignancies:
(a) 51 bone marrow (BM) samples obtained at diagnosis from acute lymphoblastic leukemia (ALL) patients
(b) 12 BM samples obtained at diagnosis from acute myeloid leukemia (AML) patients
(c) 26 primary tumors from neuroblastoma (NBL) patients

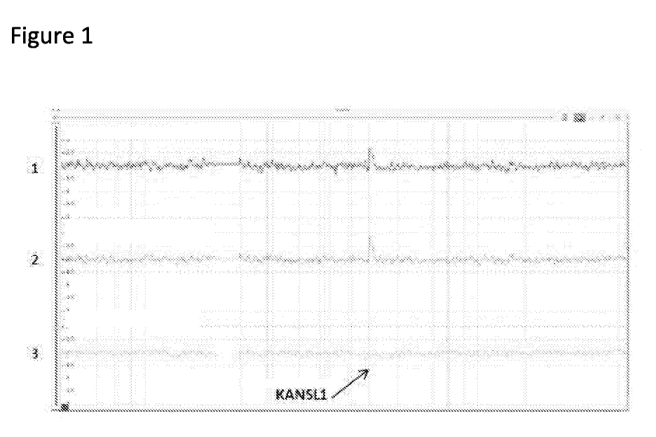

FIG. 1 shows an exemplary illustration of 17q smooth signal for three samples. The top two traces in the figure show samples having gain of KANSL1 copy number. The bottom trace shows a sample with normal KANSL1 copy number.

Gain of KANSL1 located on chromosome 17q21.31 was observed in 66% (34 out of 51) of ALL samples, in 50% (6 out of 12) of AML samples and in 85% (22 out of 26) of NBL samples. 17q gain, in general, is a known adverse prognostic marker in NBL. However, this has never before been attributed to KANSL1. Thus, following the exclusion of the samples with 17q gain, the gain of KANSL1 only (not related to 17q gain) was detected in 58% of the samples. It is notable that 7 of the ALL patients relapsed. Of these, 6 had a gain of KANSL1 copy number indicating a correlation between copy number gain and increased risk of relapse.

### Example 2

### KANSL1 Expression Validates Gain of Genomic Copy Number

This example shows that a gain of KANSL1 copy number in certain cancers correlates with increased mRNA and protein expression.

KANSL1 mRNA

To validate the KANSL1 copy number gain, KANSL1 RNA levels were measured by quantitative real time RT-PCR (RQ-PCR) using forward primer SEQ ID NO: 1 (5′ctgccaacggaaccaaaaga3′) and reverse primer SEQ ID NO: 2 (5′ctgatgtaacatctgttccc3′).

RNA was extracted from ALL, AML and NBL samples, using QIAzol lysis reagent from the miRNeasy Mini Kit (Qiagen), according to the manufacturer's protocol. First-stand cDNA synthesis from total RNA (1 μg) was performed using the quantitect-reverse transcription kit (Qiagen). Expression of target mRNA of KANSL1 was determined using quantitative RT-PCR in the LightCycler® 480 Software device. β-actin was used as control. Primers were provided from Sigma.

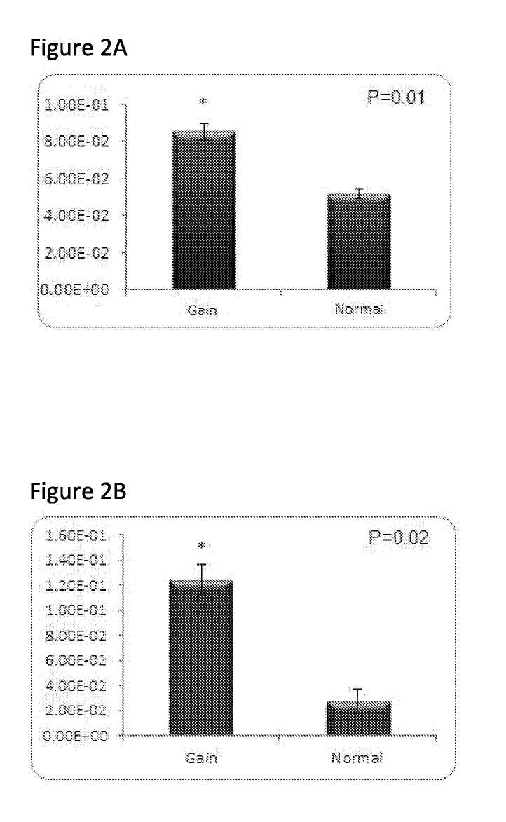

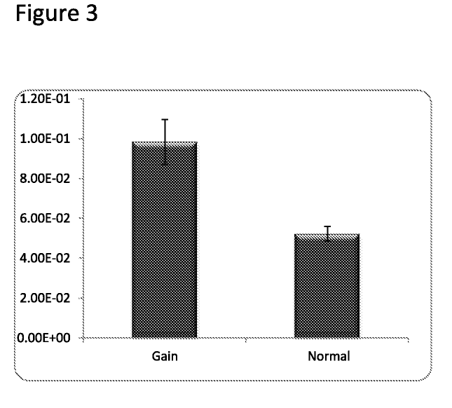

As shown in FIGS. 2A and 2B, the KANSL1 RNA levels in the samples that exhibited gain of copy number were significantly higher in comparison to the levels in the samples that exhibited normal copy number in ALL (FIG. 2A, p=0.01) and NBL (FIG. 2B, p=0.02). In the AML cohort, there was trend of higher expression levels in the group with gain of KANSL1 versus the group with normal copy number. (FIG. 3).

### KANSL1 Protein

To determine if the increased KANSL1 mRNA expression resulted in increased KANSL1 translation, protein levels were measured by Western blotting.

ALL cell extracts were electrophoresed and transferred to a membrane according to standard methods. The membrane was incubated with primary antibody against KANSL1 (Monoclonal AntiKANSL1 Abcam) and against GAPDH that served as a loading control (Monoclonal Anti-GAPDH-Santa Cruz). The membrane was read in the Micro Chemi Gel capture software using DNR device, after using Enhanced chemiluminescence (ECL) according to the manufacture's protocol (Epigenetek Inc).

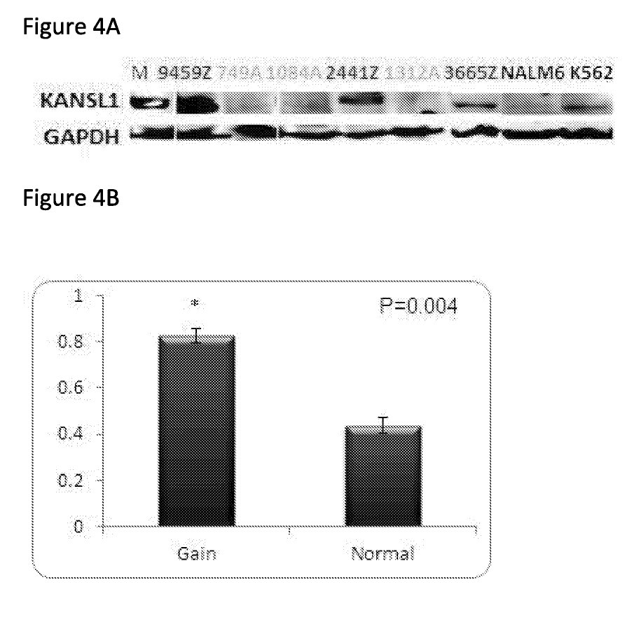

The results obtained from 19 bone marrow (BM) samples were statistically analyzed using paired T-Test. Elevated KANSL1 protein levels were identified in the samples that exhibited gain of the gene versus the levels in the samples with normal copy number (p=0.004; FIGS. 4A and 4B). In FIG. 4A, lanes ending in “Z” show samples taken from subjects having gain of KANSL1 copy number, whereas lanes ending in “A” show samples taken from subjects having normal KANSL1 copy number.

### Example 3

### Effect of KANSL1 Gain of Genomic Copy Number on Histone Acetylation

KANSL1 is part of the NSL transcriptional regulation complex, and which functions through histone acetylation. This example shows that histones of bone marrow samples from ALL patients have increased histone H3 acetylation.

Example 1 demonstrates a gain of KANSL1 copy number in bone marrow of ALL patients. To determine the effects of KANSL1 copy number gain on histone acetylation, histone acetylation was measured on histones H3 and H4 in 27 ALL patient bone marrow (BM) samples. Histones were extracted from BM samples and were analyzed for histone acetylation on different lysine residues according to manufacturer's protocol (Epigenetek Group Inc). The amount of acetylated histones were quantified through HRP (horseradish peroxidase) conjugated secondary antibody-color development system and is proportional to the intensity of color development. Results were analyzed by the absorbance on a microplate reader at 450 nm.

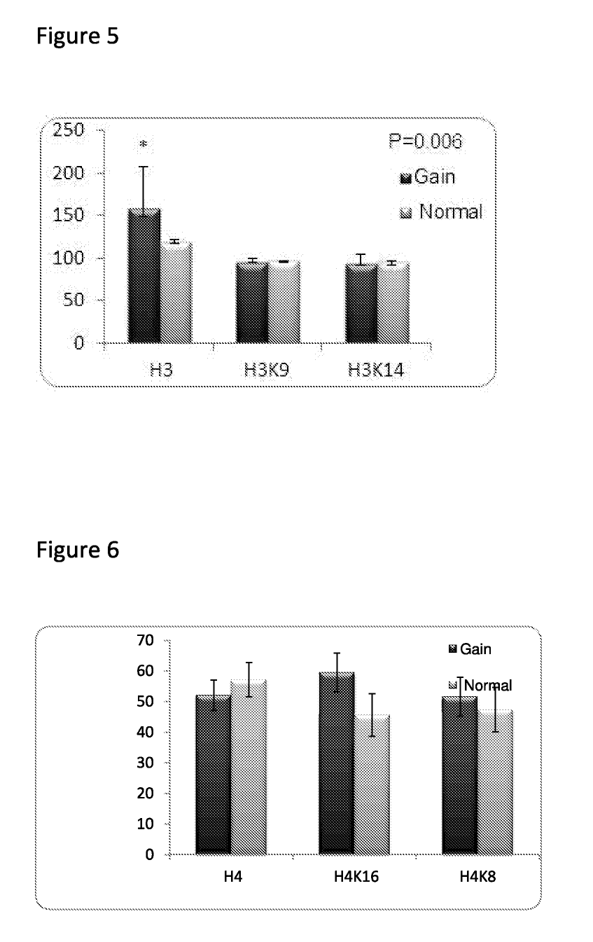

Total histone H3 acetylation levels were significantly higher (p=0.006) in patients harboring gain of KANSL1 versus those expressing normal KANSL1 copy number (FIG. 5). In the figure, “Normal” refers to ALL subjects who express normal KANSL1 copy number. Total histone H4 acetylation levels were not significantly different between samples with gain or normal copy number of KANSL1 (FIG. 6). However, a trend of higher acetylation levels was evident specifically in H4K16 (FIG. 6).

### Example 4

### Increase of MOF and TP53 Protein Expression in Bone Marrow from ALL Patients

KANSL1 works in a transcription regulatory complex with MOF, and acetylates TP53, among other targets. This example shows the investigation of MOF and TP53 levels in BM samples from ALL patients.

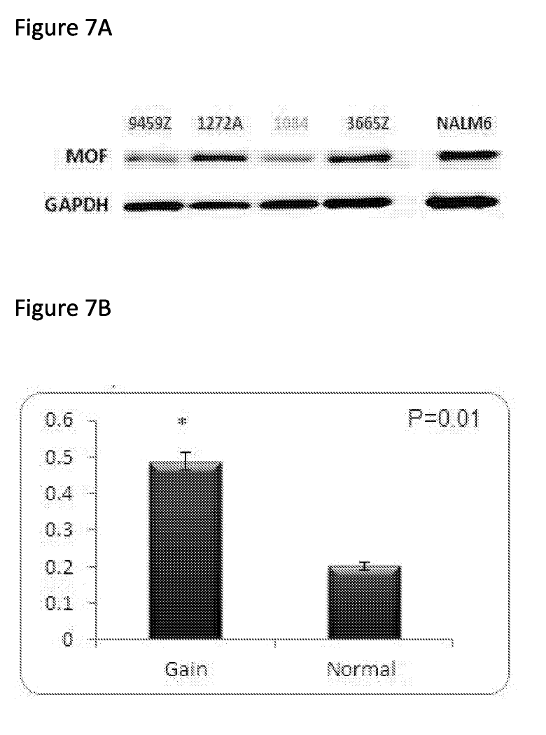

MOF protein levels were assayed in 30 BM samples from ALL patients by standard Western blotting (MOF antibody was provided by Abcam). MOF protein levels were significantly elevated in samples harboring gain of KANSL1 (p=0.01; FIGS. 7A and B) in contrast to the samples with normal KANSL1 copy number of the gene (sample 1084).

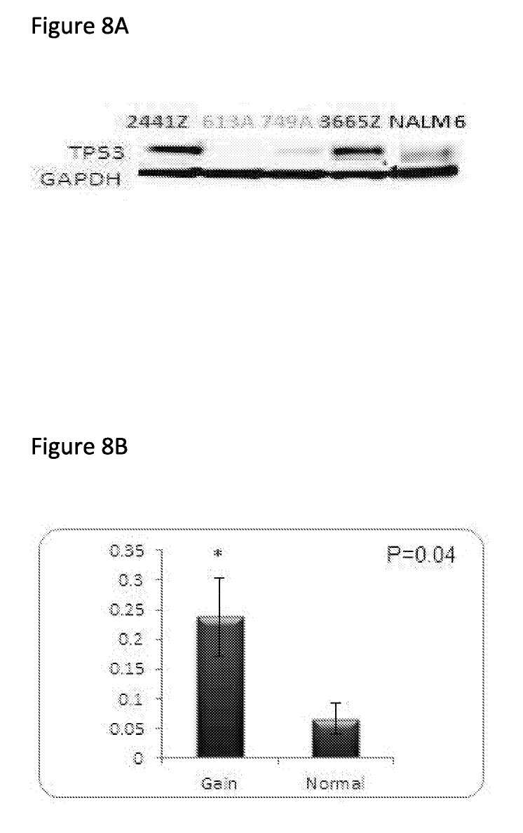

The TP53 protein was measured in 22 BM samples from ALL patients by Western Blot (antibody provided by Santa-Cruz). We identified significantly higher TP53 protein levels in patients harboring gain of KANSL1 versus the samples with normal copy number of KANSL1 (p=0.04; FIGS. 8A and 8B). In FIG. 8A, lanes ending in “Z” show samples taken from subjects having gain of KANSL1 copy number, whereas lanes ending in “A” show samples taken from subjects having normal KANSL1 copy number.

### Example 5

### KANSL1 Overexpression In Vitro Recapitulates ALL BM

This example examines the effects of overexpressing KANSL1 in a pre-B ALL cell line.

### Cell Lines

KANSL1 was overexpressed in a human pre-B ALL cell line (NALM6) using a plasmid provided by Abcam. The plasmid (2 μg) was transfected into the cells using electroporation with the Amaxa®Nucleofector technology (Lonza). Plasmid with Green Fluorescent Protein (GFP) insert (Amaxa®) was used as a negative control. Transfected NALM6 cells exhibited significantly elevated KANSL1 RNA and protein levels versus the control cells, measured by qRT-PCR and Western Blot, respectively.

### Histone H3 and H4 Acetylation in NALM6 Cells

Total Histone acetylation was measured in NALM6 cells following transfection of the KANSL1 plasmid.

Histones were extracted from NALM6 cells, and were analyzed for total histone H3/H4 acetylation (n=3) and total histone modifications (n=1) (Epigenetek Group Inc).

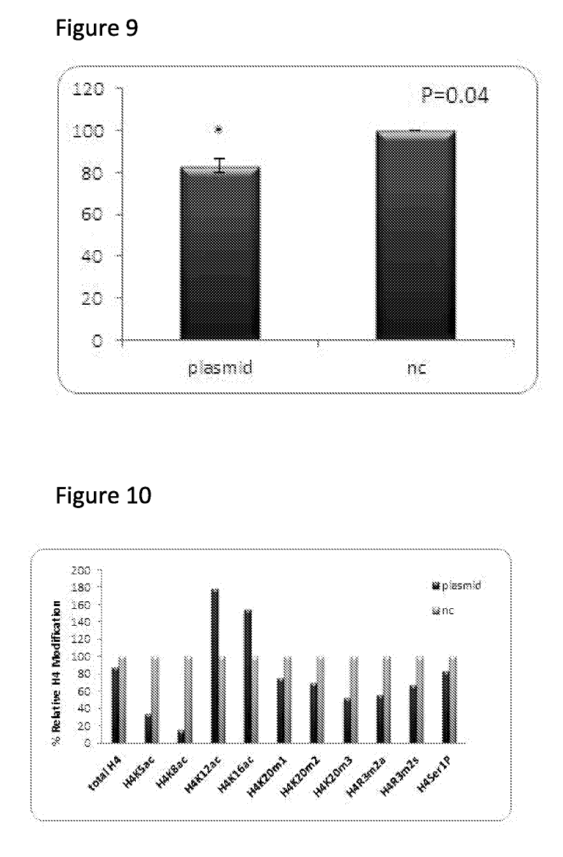

The levels of total H4 acetylation were decreased by 20% in the transfected cells versus the control cells (p=0.04, FIG. 9). Interestingly, we identified elevated levels of H4K16 and H4K12, while H4K5 and H4K8 acetylation levels were decreased in the transfected cells (FIG. 10). Elevated levels of H4K16 were also identified in the ALL patient samples.

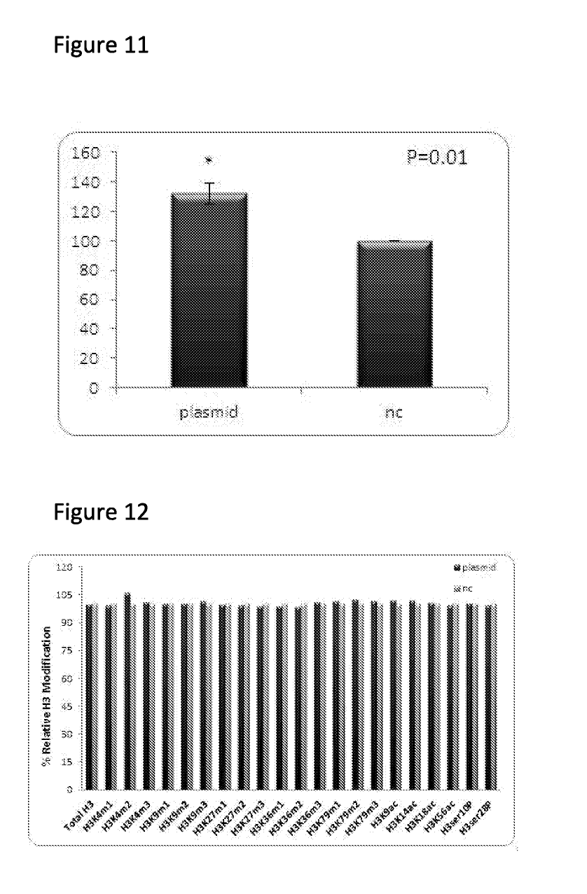

In contrast to histone H4, elevated levels of total H3 acetylation were identified in NALM6 cells following transfection (p=0.01, FIG. 11). These results correlate with the results obtained in the patients' BM samples. Elevated levels of H3 dimethyl K4 (H3K4m2) were also detected in the transfected cells (FIG. 12).

### MOF Protein Levels in NALM6 Cell Line

MOF protein was measured by Western Blot (results are from 3 independent experiments).

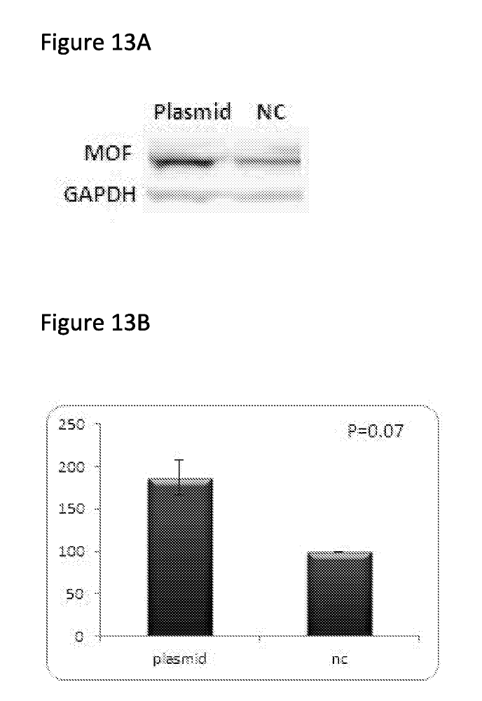

A borderline significant (p=0.07) increase in MOF levels was evident in the cells that were transfected with KANSL1 versus the control (FIGS. 13A and 13B). These results are consistent with the results from the patients' BM samples.

### TP53 Protein Levels in NALM6 Cell Line

TP53 protein was measured using Western Blot in the transfected cells (results are from 2 independent experiments).

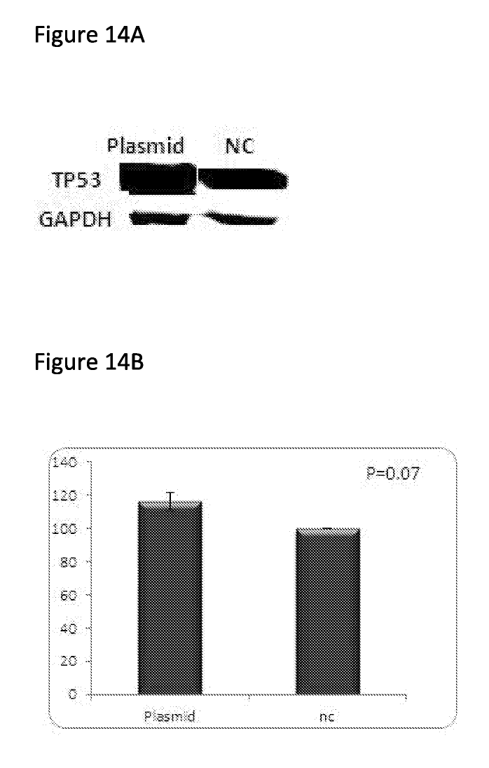

There was a borderline increase (p=0.07) of the TP53 protein levels in the transfected NALM6 cells (FIGS. 14A and 14B). These results are consistent with the results from the patients' BM samples.

### Example 6

### MG-149, a HAT Treatment-Reduces Levels of H3 Acetylation

Examples 3 and 5 demonstrate that increased KANSL1 expression, such as illustrated herein in KANSL1-associated cancers, results in aberrantly high histone H3 acetylation. This example shows that in vitro exposure of cells overexpressing KANSL1 to the HAT inhibitor MG-149 will reduce aberrant H3 acetylation to near control levels.

NALM6 cell line was grown in RPMI (ATCC) supplemented with 10% fetal bovine serum (FBS) (Life Technologies), 0.1% L-Glutamine and 0.1% Penicillin Streptomycin (PS) (Life Technologies). Cells were cultured in a humidified atmosphere in an incubator at 37° C. with 5% CO₂. Plasmid overexpression transfection was done by electroporation, using the Amaxa® Nucleofector technology (Lonza). Purified plasmid was sent to Maxi-Prep (Hy-Labs) and a concentration of 2 μg was taken for the transfection. As a negative control, a Green Fluorescent Protein (GFP) plasmid provided by the Amaxa kit was used.

Cells were treated with MG-149 (a novel anacardic acid that demonstrates selective inhibition of the MYST type of histone acetyltransferase, Dekker et al) at an IC50 of 20 μM, 24 h after the transfection. Histones were extracted 48 h after transfection. Total histone protein concentration was adjusted to 400 ng/μl. Histone acetylation quantification was analyzed according to the manufacture's protocol (Epigentek).

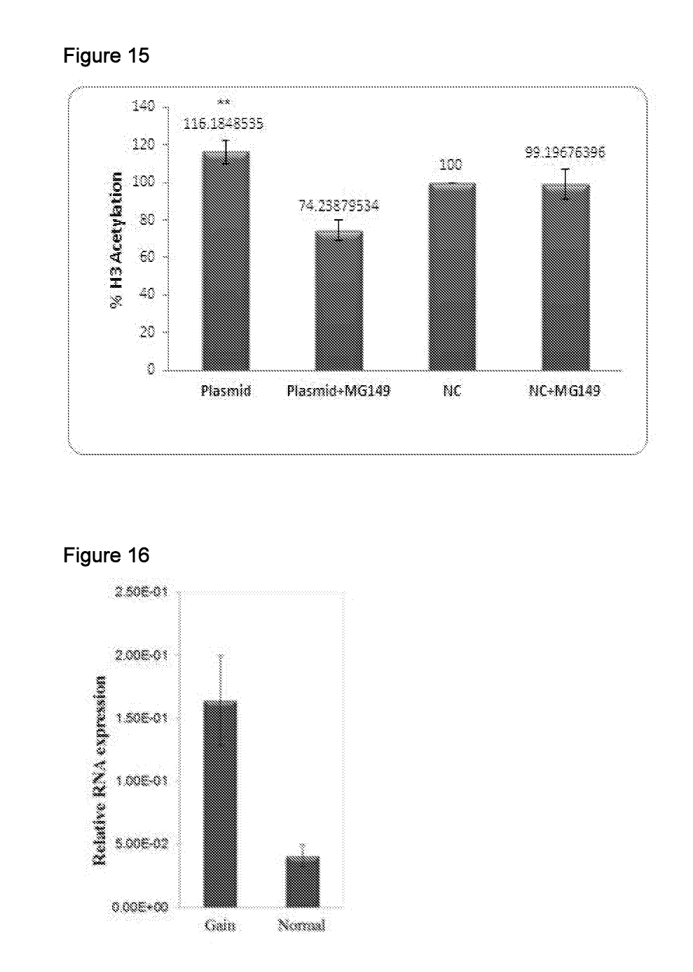

As shown in FIG. 15, when exposed to MG-149, the aberrantly high acetylation of histone H3 in cells overexpressing KANSL1 is inhibited to near control levels. This experiment is proof of concept that MG-149 (as an exemplary HAT specific inhibitor) may be used as a drug in patients overexpressing KANSL1, thereby treating a KANSL1-associated cancer.

### Example 7

### Increase of KANSL1 Copy Number in Ependymoma and Ewing Sarcoma Patients

As described above, several cancers were identified as being strongly associated with a gain of KANSL1 copy number. This example broadens this correlation, indicating that a gain of KANSL1 copy number, where detected, strongly suggests a pre-disposition to cancer development and indicates a need for treatments to inhibit relapse of a patient undergoing treatment.

KANSL1 copy number was analyzed in 34 primary and 10 relapse ependymoma tumor samples by Cytoscan HD SNP array (Affymetrix) according to manufacturer's instructions. Gain of KANSL1 was detected in 18 (53%) of primary tumors and 90% of relapse samples.

KANSL1 gain in ependymoma samples was validated by RQ-PCR. mRNA expression levels of KANSL1 was measured in 27 primary ependymoma samples as described above. Fourteen of the samples had KANSL1 copy number gain and 13 normal copy number. As shown in FIG. 16, a significant correlation between copy number gain and high expression levels was identified (p=0.0046).

Similarly, KANSL1 copy number was analyzed in 6 primary and 6 relapse Ewing sarcoma tumor samples by Cytoscan HD SNP array (Affymetrix) according to manufacturer's instructions. Gain of KANSL1 was detected in 50% of primary and 50% of relapse samples.

### REFERENCES

Cohen I, Por [symbol] ba E, Kamieniarz K and Schneider R. Histone Modifiers in Cancer, Friends or Foes? Genes and Cancer: 2(6); 631-647, 2011.
Dekker F J, van den Bosch T, and Martin N I. Small molecule inhibitors of histone acetyltransferases and deacetylases are potential drugs for inflammatory diseases, Drug Discovery: Vol 19 (5);654-660, 2014.
Ghizzoni M, Wu J, Gao T, et al. 6-alkylsalicylates are selective Tip60 inhibitors and target the acetyl-CoA binding site, European Journal of Medicinal Chemistry: Vol 47: 337-344; 2011
Legartová S, Stixová L, Strnad H, et al. Basic nuclear processes affected by histone acetyltransferases and histone deacetylase inhibitors, Future Medicine: Vol 5 (4); 379-396, 2013.
Kadlec J, Hallacli E, Lipp M, et al. Structural basis for MOF and MSL3 recruitment into the dosage compensation complex by MSL1. Nat Struct Mol Biol 18: 142-149, 2011.
Li X and Dou Y, “New Perspectives for the Regulation of Acetyltransferase MOF”, Epigenetics, 2010; Vol 5(3): 185-188.
Rosas A H, Rojas M D, Arceo S D B, et al. Effect of 6-nonadecyl salicylic acid and its methyl ester on the induction of micronuclei in polychromatic erythrocytes in mouse peripheral blood Mutation Research/Genetic Toxicology and Environmental Mutagenesis: Vol 609 (1); 43-46, 2006.
Shahbazian M D, Grunstein M. Functions of site-specific histone acetylation and deacetylation. Annu Revf Biochem 76:75-100, 2007.
Shehzad A, Jaetae Lee J, and Lee Y S, “Curcumin in various Cancers”, BioFactors: Vol 39 (1); 56-68, 2013.
Taipale M, Rea S, Richter K, et al. hMOF histone acetyltransferase is required for histone H4 lysine 16 acetylation in mammalian cells. Mol Cell Biol 25: 6798-6810, 2005.
Thomas T, Dixon M P, Kueh A J, Voss A K. 2008. Mof (MYST1 or KAT8) is essential for progression of embryonic development past the blastocyst stage and required for normal chromatin architecture. Mol Cell Biol 28: 5093-5105, 2008.
Yoshida K, Told T, Okuno Y, et al. The landscape of somatic mutations in Down syndrome related myeloid disorders. Nat Genet 44: 1293-1299, 2013.
Zollino M, Orteschi D, Murdolo M, et al. Mutations in KANSL1 cause the 17q21.31 microdeletion syndrome phenotype. Nat Genet 44: 636-638, 2012.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims

We claim:

1. A method for diagnosing a cancer, predicting a predisposition to a cancer and/or predicting an appropriate epigenetic therapy for a cancer, the method comprising:
detecting KANSL1 expression and/or copy number in a sample from a subject, wherein a measurable increase in KANSL1 expression and/or copy number when compared with a control diagnoses, predicts the predisposition, and/or predicts the appropriate epigenetic therapy for the cancer in the subject.

2. The method of claim 1, wherein the cancer is selected from the group consisting of ALL, AML, Ependymoma. Ewing sarcoma, and neuroblastoma.

3. The method of claim 1 or claim 2, wherein detecting KANSL1 expression comprises detecting KANSL1 RNA or protein in the sample.

4. The method of any one of claims 1-3, wherein the subject is a human or a non-human subject.

5. A method of determining the prognosis and the therapy of a cancer comprising:
detecting KANSL1 copy number and/or expression in a sample from a subject diagnosed with the cancer, and wherein a significant increase in KANSL1 copy number and/or expression compared with a control indicates an increased probability that the subject will relapse.

6. The method of claim 5 wherein the cancer is selected from the group consisting of ALL, AML, Ependymoma, Ewing sarcoma, and neuroblastoma.

7. The method of claim 5 or claim 6, wherein detecting KANSL1 expression comprises detecting KANSL1 RNA or protein in the sample.

8. A method of treating a cancer, comprising:
administering to a subject in need thereof a therapeutically effective amount of an inhibitor of KANSL1 activity, thereby treating the cancer.

9. The method of claim 8, wherein the cancer is selected from a group consisting of ALL, AML, Ependymoma. Ewing sarcoma, and neuroblastoma.

10. The method of claim 8, wherein the inhibitor blocks formation of the NSL transcriptional regulation complex.

11. The method of claim 8, wherein the inhibitor blocks the HAT activity of the NSL complex.

12. The method of claim 8, wherein the inhibitor specifically binds to KANSL1.

13. The method of claim 8, wherein the inhibitor specifically binds to a non-KANSL1 member of the NSL complex.

14. The method of claim 13, wherein the non-KANSL1 member of the NSL complex is MOF.

15. The method of claim 8, wherein the inhibitor transiently inhibits the expression of KANSL1.

16. The method of claim 15, wherein the inhibitor is an RNAi agent.

17. The method of claim 8, wherein the inhibitor is an HDAC.

18. The method of claim 8, wherein the inhibitor comprises MG-149.

19. The method of any one of claims 1-7, wherein the methods indicate a need to inhibit NSL complex activity, and further comprising administration of an inhibitor of KANSL1 activity.

20. The method of claim 19, wherein the KANSL1 inhibitor is an inhibitor of HAT activity of the NSL complex.

21. The method of claim 19, wherein the KANSL1 inhibitor is an HDAC.

22. The method of claim 19, wherein the inhibitor comprises MG-149.


SEQUENCE LISTING (4 sequences)

SEQ ID NO: 1 - DNA, 20 bp, Artificial Sequence
  Synthetic oligonucleotide
ctgccaacggaaccaaaaga

SEQ ID NO: 2 - DNA, 20 bp, Artificial Sequence
  Synthethic oligonucleotide
ctgatgtaacatctgttccc

SEQ ID NO: 3 - DNA, 20 bp, Artificial Sequence
  Synthetic oligonucleotide
catgtacgttgctatccagg

SEQ ID NO: 4 - DNA, 20 bp, Artificial Sequence
  Synthetic oligonucleotide
ctcgtagatgggcacagtgt